(19) United States
(12) Patent Application Publication
(10) Pub. No.: US 2011/0086347 A1
(43) Pub. Date: Apr. 14, 2011

(54) CELL DETECTION, MONITORING AND ISOLATION METHOD

(75) Inventors: Damien Luis Bruno, Keilor (AU); Howard Robert Slater, Parkville (AU)

(73) Assignee: MURDOCH CHILDRENS RESEARCH INSTITUTE, Parkville (AU)

(21) Appl. No.: 12/680,415

(22) PCT Filed: Sep. 26, 2008
(86) PCT No.: PCT/AU2008/001440

§ 371 Date: Dec. 28, 2010

(30) Foreign Application Priority Data
Sep. 28, 2007 (AU) 2007905324

(51) Int. Cl. C12Q 1/68

(52) U.S. Cl. 435/6

(57) ABSTRACT

The present invention relates generally to a method for identifying or distinguishing one type of cell from other cells within a population of cells. The present invention further provides the detection, monitoring and isolation of sub-populations cell types within a population of cells including a biological entity comprising such cell types. Kits, diagnostic agents and panels of nucleic acid probes for identifying and distinguishing cell types also form part of the present invention. The detection of particular cell types is based upon the use of a labelled probe that hybridizes to chromosomal DNA at the flanking sequences of a deletion. Alternatively two probes with distinguishable reporter molecules are used wherein only one probe is capable of hybridizing to chromosomal DNA in one cell type whereas both are capable of hybridizing to chromosomal DNA in another cell type. The methods are useful in the identification of cells with copy number variations, chromosomal deletions, additions or aberrations.

### RELATED APPLICATION DATA

This application is associated with and claims priority from Australian Patent Application No. 2007905324, filed on 28 Sep. 2007, the content of which is incorporated herein in its entirety by reference.

### FIELD

The present invention relates generally to a method for identifying or distinguishing one type of cell from other cells within a population of cells. The present invention further provides the detection, monitoring and isolation of sub-populations cell types within a population of cells including a biological entity comprising such cell types. Kits, diagnostic agents and panels of nucleic acid probes for identifying and distinguishing cell types or for nucleic acid probes useful for same also form part of the present invention.

### BACKGROUND

Reference to any prior art in this specification is not, and should not be taken as, an acknowledgment or any form of suggestion that this prior art forms part of the common general knowledge in any country.

Genetic testing has the potential to provide a highly sensitive approach to identifying genetic disorders. However, this approach can be invasive, particularly in the prenatal setting. When testing for fetal genetic abnormalities (e.g. Down Syndrome), invasive sampling (i.e. by amniocentesis or chorionic villus) may be required which involves the aspiration of a small sample of material (amniotic fluid or placental tissue) from the pregnant mother, culturing fetal cells from the fluid and then determining the karyotype of the fetal cells. Non invasive tests for determining fetal genetic abnormalities are impeded by the high proportion of maternal cells to fetal cells.

The rapid and sensitive detection of particular cell types in a high through put manner would be advantageous. This would allow ready screening not only of fetal cells but a range of cell types such as transplanted cells, cancer cells and other potentially rare cell types which can then be subjected to genetic testing.

There is a need to develop such a cell detection, monitoring and isolation technique.

### SUMMARY

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention is predicated in part on the identification, monitoring and/or isolation of particular cell types within a population of cells based on nucleic acid alterations in chromosomes. Such “cell types” are regarded herein as a sub-population of cells or cell types within a population of cells. Particular cell types are identified by a copy number variation (CNV) polymorphism which is a characteristic of the chromosomal DNA of the targeted cell type. Hence, as indicated above, the target cell type is a sub-population of cells within a population of cells. The sub-population may be a rare cell type such as a cell type arising at a frequency of from 1×10⁻¹⁴ to 1×10⁻⁶ such as 1×10⁻¹² to 1×10⁻⁷, for example, 1×10⁻⁸. However, in some cases, such as cancer, where cancer is rampant, the frequency might be much higher and in fact a “normal” cell may be in the minority. Reference to a “rare” or “minor” cell generally means a cell occurring at a frequency of from 1×10⁻¹⁴ to 1×10⁻⁶ but encompasses any cell which occurs at a frequency less than the predominant or most common cell. The sub-population of cells may, therefore, also occur at a much higher frequency. Cells are detected on the basis of the presence or absence of a CNV deletion in a cell type. The CNV may also itself be associated with a genetic disorder or predisposition for developing same, or a further test is conducted to detect chromosomal alterations characteristic of a genetic disorder or predisposition for developing same. The CNV may also merely be characteristic of the sub-population of cells or general cell type. Hence, the present invention uses CNV polymorphisms as a tag to identify, monitor and, if necessary, isolate cells of a particular cell type within a population of cells.

In one type of nucleic acid probe-based assay of cell types, a CNV polymorphism is detected using a probe which binds to the junction formed in the targeted cell's chromosomal DNA following a deletion. A targeted cell is detected on the basis of a deletion which is present in its chromosome but absent in the chromosomal DNA of the other cells of the population. In one embodiment, only the targeted chromosome will have the nucleotide sequence formed at the junction of DNA after a deletion. In these assay types, a single probe labeled with a reporter molecule provides a single signal in a targeted cell when the probe binds to chromosomal DNA of the cell. The probe will not bind to other chromosomal DNA derived from other cells in the population, due to the CNV.

In another type, two probes are employed each labeled with a reporter molecule capable of giving a different or distinguishable signal. The probes are selected such that one has a complementary DNA sequence to a chromosomal DNA flanking or adjacent to a deletion in a corresponding chromosome of one cell type, while the other has a complementary DNA sequence to all or part of the sequence which has been deleted in a chromosome of another cell type. In this case, both probes bind to DNA of one cell type giving a combination of two signals from the reporter molecules but only one probe binds to a chromosomal DNA with a deletion in another cell type. Hence, one cell type can be distinguished from another cell type based on a combination of the two signals or a single signal.

In another type a single probe is used labeled with a reporter molecule which targets a copy number deletion present in the non-rare cell type that is not present in the target cell type. In this case, an extra signal is present in cells that do not harbour the copy number deletion (i.e. rare cell type). Hence, one cell type can be distinguished from another cell type based on the presence of an addition signal.

One aspect of the present invention contemplates a method for identifying a cell type in a sample comprising a mixture of cell types from a subject, the method comprising collecting the sample with the cells and contacting the cells with a nucleic acid probe labeled with a reporter molecule capable of giving an identifiable signal which probe is capable of hybridizing to chromosomal DNA at the flanking sequences of a deletion in one cell type, which deletion absent in chromosomal DNA in another cell type, wherein the presence or absence of a signal in a cell is indicative of a particular cell type.

Reference to a “signal” includes a combination of signals. A “combination” might result, for example, in a color being generated by two other colors.

Another aspect of the present invention provides a method for identifying a cell type in a sample comprising a mixture of cell types from a subject, the method comprising collecting the sample with the cells and contacting the cells with two nucleic acid probes each labeled with distinguishable reporter molecules each capable of giving an identifiable signal wherein one nucleic acid probe is capable of hybridizing to chromosomal DNA from one cell type whereas both nucleic acid probes are capable of hybridizing to a portion of chromosomal DNA present in another cell type, wherein the presence or absence of a single signal or a combination of signals in a cell is indicative of the cell type.

Generally, the probe is identifying a copy number variation (CNV) which includes a copy number deletion or insertion.

The methods and assays of the present invention may be automated or semi-automated and may additionally employ sorting by FACS. Robotic screening of cells such as via a scanning microscope also forms part of the present invention. Reference to “robotic screening” includes any form of automation generally interfaced with software. High frequency throughput assays are contemplated herein as are any form of visual detection means.

In an embodiment, the sample with the cells is distributed onto a solid support. This aspect is directed to microscopic screening rather than FACS. Microscopic screening includes the use of microscopy or other visual detection aids or devices.

The present invention further contemplates a method for sorting or identifying a cell type in a sample from a mixture comprising other cell types from a subject, the method comprising collecting the sample with the cells and contacting the cells with a nucleic acid probe labeled with a reporter molecule capable of giving an identifiable signal which probe is capable of hybridizing to chromosomal DNA at the flanking sequences of a deletion in one cell type, which deletion is absent in chromosomal DNA of another cell type, wherein the presence or absence of a signal in a cell is indicative of a particular cell type, the method further comprising subjecting the cells to FACS or microscopy or other visual detection means.

Another aspect of the present invention provides a method for sorting or identifying a cell type in a sample from a mixture comprising other cell types from a pregnant female subject, the method comprising collecting the sample with the cells contacting the cells with two nucleic acid probes each labeled with distinguishable reporter molecules each capable of giving an identifiable signal wherein one nucleic acid probe is capable of hybridizing to chromosomal DNA in one cell type whereas both nucleic acid probes are capable of hybridizing to a portion of chromosomal DNA present in another cell type; wherein the presence or absence of a single signal or a combination of signals in a cell is indicative of the cell type being identified cell, the method further comprising subjecting the cells to FACS or microscopy or other visual detection means.

Again, in a particular aspect, the probes identify the presence or absence of CNV (which includes a copy number deletion or insertion).

Kits comprising nucleic acid probes, solid supports and/or compartments suitable for conducting the assays are also contemplated herein. For microscopic screening, the cells may be distributed onto a solid support. Microscopic includes microscopy.

A panel of probes covering particular known CNVs in normal cells or abnormal cells such as cancer cells, diseased neurological cells, organ- or tissue-specific cells is also contemplated herein. The panel could be used, for example, to determine which probe or collection of probes will be used to screen particular subjects (e.g. transplant subjects, subjects with particular cancers, etc). Hence, such a panel is useful, for example, in monitoring for cancer cells in subjects, monitoring the effectiveness of cancer treatments, monitoring donor transplanted cells within a population of recipient cells and monitoring for fetal cells within a population of maternal cells.

The ability to detect certain cell types (sub-populations) of cells within a larger population of cells enables monitoring and scanning for, in an example, rare cells such as cancer cells, transplanted cells such as bone marrow transplanted cells, fetal cells within a population of maternal cells and so on. In addition, once a particular cell is identified, it can then be subjected to further testing such as genetic testing.

A range of genetic disorders may, therefore, be screened including aneuploidy (e.g. trisomy associated with Down Syndrome or Turner Syndrome), polyploidy (e.g. triploidy such as associated with whole cell 69 chromosomes), or any syndrome where an established aetiology of segmented copy number abnormality (e.g. Prader Willi Syndrome). Cell fate after transplantation can also be determined.

Furthermore, the ability to detect rare cell types facilitates methods of treatment involving rare cells. For example, autologous or heterologous stem cells may be identified and the population expanded ex vivo before being re-introduced to the subject or another subject. In addition, using the CNV signature of the present invention, the fate and/or distribution of these stem cells can be followed and monitored.

Hence the present invention contemplates a method of treatment or monitoring a treatment, the method comprising collecting a sample comprising cells to be transplanted to a subject and contacting the cells with a nucleic acid probe labeled with a reporter molecule capable of giving an identifiable signal which probe is capable of hybridizing to chromosomal DNA at the flanking sequences of a deletion in one cell type, which deletion absent in chromosomal DNA in another cell type, wherein the presence or absence of a signal in a cell is indicative of a particular cell type expanding the desired cells ex vivo and then transplanting the cells into the same subject or a different subject and then using the same method to monitor distribution of said cells.

Another aspect of the present invention provides a method of treatment or monitoring a treatment, the method comprising collecting a sample comprising cells to be transplanted to a subject, contacting the cells with two nucleic acid probes each labeled with distinguishable reporter molecules each capable of giving an identifiable signal wherein one nucleic acid probe is capable of hybridizing to chromosomal DNA from one cell type whereas both nucleic acid probes are capable of hybridizing to a portion of chromosomal DNA present in another cell type, wherein the presence or absence of a single signal or a combination of signals in a cell is indicative of the cell types expanding the desired cells ex vivo and then transplanting the cells into the same subject or a different subject and then using the same method to monitor distribution of said cells.

Generally, the desired cells are stem cells. In a particular embodiment, the stem cells are autologous to the subject being treated. The subject may be a human or non-human animal.

### BRIEF DESCRIPTION OF THE FIGURE

Some figures contain color representations or entities. Color photographs are available from the Patentee upon request or from an appropriate Patent Office. A fee may be imposed if obtained from a Patent Office.

FIG. 1 is a graphical representation of 131 of the most common copy number deletions (Database of Genomic Variants, September 2008). Each point represents an individual CND of a given size (X-axis) and frequency (Y-axis). The average study size (i.e. number of normal individuals tested) varied from 20 to 270 (mean of 140). Data obtained from the Database of Genomic Variants (http://projects.tcag.ca/variation/).

FIG. 2 is a schematic diagram of the assay for Down Syndrome.

FIG. 3 is a graphical representation of a copy number deletion in chromosome 21 detected in the DNA of a child with Down Syndrome using SNP microarray analysis. The deletion is 200 kb in size.

FIG. 4 is a photographic representation of a deletion within two cell nuclei using Dual FISH Probe method.

FIG. 5 is a diagrammatic representation showing location of FISH probes for detection of copy number deletions (CND-FISH).

FIGS. 6a and b are photographic representations showing spiking experiments using mixtures of lymphocyte cells from a child with Down syndrome and his mother. An example of a paternal-specific 50 Kb copy number deletion in a child with Down syndrome. FISH probes within and flanking the deletion have been designed and hybridized to mixtures of the child's and mother's lymphocytes. The mother (b) and child's (a) cells are distinguished by the number of combination signals (2 and 1, respectively) and the enumeration made using chromosome 21-specific probes.

FIG. 7 is a graphical representation of genome screening using 250 K Nsp SNP arrays. Identification of a 50 Kb deletion (AROMA), on chromosome 9p23.1, in the DNA from a child with Down syndrome and his father.

### DETAILED DESCRIPTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

As used in the subject specification, the singular forms “a”, “an” and “the” include plural aspects unless the context clearly dictates otherwise. Thus, for example, reference to “a cell” includes a single cell, as well as two or more cells; reference to “an assay” including a single assay as well as two or more assays; reference to “the invention” includes a single or multiple aspects of the invention; and so on.

The term “subject” as used herein refers to an animal, particularly a mammal and more particularly a primate including a lower primate and even more particularly a human who can benefit from the methods and assays of the present invention. A subject regardless of whether a human or non-human animal or embryo may be referred to as an individual, subject, animal, patient, host or recipient. Although the present assay is particularly applicable to identifying human cells or various types of human cells, it is also applicable to identifying non-human cells. The present invention, therefore, has both human and veterinary applications. For convenience, an “animal” specifically includes livestock animals such as cattle, horses, sheep, pigs, camels, goats and donkeys. With respect to horses, these include horses used in the racing industry as well as those used recreationally or in the livestock industry.

Examples of laboratory test animals include mice, rats, rabbits, guinea pigs and hamsters. Rabbits and rodent animals, such as rats and mice, provide a convenient test system or animal model as do primates and lower primates.

The “subject” is regarded as comprising a population of different cell types. The present invention enables a distinction to be made between a group or sub-population of cells or cell types within the population of cells. The sub-population may be from another host such as donor versus recipient cells in a transplant situation or fetal versus maternal cells in a pregnant female or cancer versus non-cancer cells in a subject. The cells to be targeted for identification, monitoring or isolation may be the “sub-population” of cells (e.g. particularly rare cells) or the non-sub-population of cells (e.g. recipient as opposed to donor cells or maternal as opposed to fetal cells).

The present invention use a genetic basis in cell discrimination techniques to identify, monitor and optionally isolate a targeted cell type. As indicated above, the targeted cell type may be a rare cell type in a larger cell population or it may be the predominant cell type. In a particular embodiment, the present invention uses CNV polymorphisms such as deletion CNV polymorphisms to stratify cells on the basis of the polymorphism (presence or absence) to discriminate between one cell type from another. The CNV may also be associated with a particular genetic disorder. A “CNV” includes a copy number deletion and a copy number insertion.

The terms “sub-population” and “cell type” are used interchangeably herein to refer to the target cells. A cell type includes, however, the predominant cell type in a population of cells.

The identified cells may be sorted by FACS procedures using the differential signals from the reporter molecules or the signal produced by a combination of signals. This aspect of the present invention may, therefore, be automated or semi-automated. Scanning microscopy using robotics forms part of this aspect of the present invention.

Accordingly, one aspect of the present invention contemplates a method for identifying a cell type in a sample comprising a mixture of cell types from a subject, the method comprising collecting the sample with the cells and contacting the cells with a nucleic acid probe labeled with a reporter molecule capable of giving an identifiable signal which probe is capable of hybridizing to chromosomal DNA at the flanking sequences of a deletion in one cell type, which deletion absent in chromosomal DNA in another cell type, wherein the presence or absence of a signal in a cell is indicative of a particular cell type.

Reference to a “signal” includes a combination of signals. A “combination” might result, for example, in a color being generated by two other colors.

Another aspect of the present invention provides a method for identifying a cell type in a sample comprising a mixture of cell types from a subject, the method comprising collecting the sample with the cells and contacting the cells with two nucleic acid probes each labeled with distinguishable reporter molecules each capable of giving an identifiable signal wherein one nucleic acid probe is capable of hybridizing to chromosomal DNA from one cell type whereas both nucleic acid probes are capable of hybridizing to a portion of chromosomal DNA present in another cell type, wherein the presence or absence of a single signal or a combination of signals in a cell is indicative of the cell type.

Generally, the probe is identifying a copy number variation (CNV) which includes a copy number deletion or insertion.

Hence, the sample with the cells may also be distributed to a solid support prior to application of the probes. This is useful for microscopy or other visual detection means rather than FACS.

Reference to a “solid support” includes a planar surface such as a microscope slide, petri dish or other solid support made from glass, plastic, polyethylene support or other transparent or semi-transparent material. The solid support may also be part of a kit or apparatus.

In a particular embodiment, the cells are fixed to the planar surface of the solid support or are maintained in position by a cover slip or other planar surface.

One use of cells identified by the present method of the present invention is for subsequent genetic analysis, biochemical analysis, immunological analysis, morphological analysis, histology, cytology, cell culture and the like. The method of the present invention is also useful for monitoring cells such as transplanted cells, for example, following a bone marrow transplantation. The final or transient destination of these cells may be of therapeutic or physioepidermiological use. Other cell types include monitoring for cancer cells or particular cell types such as sub-populations of stem cells, immune cells and neurological cells.

A particular use is for genetic analysis of cells including fetal cells. The fetal cells may be of human or animal origin.

As used herein, “genetic analysis” and “genetic diagnosis” are used interchangeably and broadly cover detection, analysis, identification and/or characterization of genetic material and includes and encompasses terms such as, but not limited to, genetic identification, genetic diagnosis, genetic screening, genotyping, cancer cell identification, pre-natal genetic diagnosis, paternity testing and DNA fingerprinting which are variously used through this specification.

Genetic testing to assess the presence of CNV polymorphisms in the cells or other chromosomal mutations or alterations in chromosome number such as in aneuploidy (e.g. trisomy associated with Down Syndrome or Turner Syndrome), polyploidy (e.g. triploidy such as associated with whole cell 69 chromosomes), or any syndrome where an established aetiology of segmented copy number abnormality (e.g. Prader With Syndrome). A CNV also encompasses a copy number deletion and insertion.

Hence, another aspect of the present invention is also directed to an assay to detect a potential genetic abnormality in cells, the method comprising:

(i) obtaining a sample from a subject to be tested comprising both normal cells and abnormal cells;
(ii) optionally distributing the cells on a solid support if there is a microscopic component in the assay;
(iii) contacting the cells with a nucleic acid probe labeled with a reporter molecule capable of giving an identifiable signal which probe is capable of hybridizing to derived chromosomal DNA at the flanking sequences of a deletion, which deletion in a normal or abnormal cell is absent in the chromosomal DNA of the other of the abnormal or normal cell;
(iv) determining whether the cells are normal or abnormal based on the presence of a cell exhibiting or not exhibiting a signal; and
(v) conducting a genetic test on the chromosomes in the selected cells including determining a CNV profile associated with a genetic disorder or a predisposition of developing same.

The present invention is also directed to an assay to detect a potential genetic abnormality in cells, the method comprising:

(i) obtaining a sample from a sample comprising both normal and abnormal cells;
(ii) optionally distributing the cells on a solid support if there is a microscopic component in the assay;
(iii) contacting the cells with two nucleic acid probes each labeled with distinguishable reporter molecules each capable of giving an identifiable signal wherein one nucleic acid probe is capable of hybridizing to both normal and abnormal chromosomal DNA and the other nucleic acid probe is capable of hybridizing to a portion of chromosomal DNA present in one or other of the chromosomes of a normal or abnormal cell;
(iv) determining whether a cell is a normal or abnormal cell based on the presence of a cell exhibiting a single signal or a combination of signals; and
(v) conducting a genetic test on the chromosomes in the selected cells or determining a CNV profile associated with a genetic disorder or a predisposition of developing same.

In an embodiment, the present invention is directed to analysing fetal cells.

Hence, another aspect of the present invention is directed to an assay to detect a potential genetic abnormality in fetal cells, the method comprising:

(i) obtaining a sample from a pregnant female comprising both maternal and fetal cells;
(ii) optionally distributing the cells on a solid support if there is a microscopic component in the assay;
(iii) contacting the cells with a nucleic acid probe labeled with a reporter molecule capable of giving an identifiable signal which probe is capable of hybridizing to paternally-derived chromosomal DNA at the flanking sequences of a deletion, which deletion is absent in maternal chromosomal DNA;
(iv) determining whether the cells is a fetal or maternal cell based on the presence of a cell exhibiting a signal wherein a signal represents a fetal cell; and
(v) conducting a genetic test on the chromosomes in the fetal cells or enumerating the chromosomes in the fetal cells or determining a CNV profile associated with a genetic disorder or a predisposition of developing same.

The CNV may alternatively be a deletion in the maternal chromosome.

Hence, a related embodiment is directed to an assay to detect a potential genetic abnormality in fetal cells said method comprising:

(i) obtaining a sample from a pregnant female comprising both maternal and fetal cells;
(ii) optionally distributing the cells on a solid support if there is a microscopic component in the assay;
(iii) contacting the cells with a nucleic acid probe labeled with a reporter molecule capable of giving an identifiable signal which probe is capable of hybridizing to paternally-derived chromosomal DNA within a sequence which is deleted in maternal chromosome DNA;
(iv) determining whether the cells is a fetal or maternal cell based on the presence of a cell exhibiting a signal wherein the presence of two signals is indicative of a fetal cell; and
(v) conducting a genetic test on the chromosomes in the fetal cells or enumerating the chromosomes in the fetal cells or determining a CNV profile associated with a genetic disorder or a predisposition of developing same.

The present invention is also directed to an assay to detect a potential genetic abnormality in fetal cells, the method comprising:

(i) obtaining a sample from a pregnant female comprising both maternal and fetal cells;
(ii) optionally distributing the cells on a solid support if there is a microscopic component in the assay;
(iii) contacting the cells with two nucleic acid probes each labeled with distinguishable reporter molecules each capable of giving an identifiable signal wherein one probe is capable of hybridizing to both maternal and paternal chromosomal DNA and the other probe is capable of hybridizing to a portion of chromosomal DNA present in the maternal chromosome but deleted in the paternal chromosome;
(iv) determining whether a cell is a fetal or maternal cell based on the presence of a cell exhibiting a single signal together with a combination of signals wherein a fetal cell will exhibit a single signal; and
(v) conducting a genetic test on the chromosomes in the fetal cells or enumerating the chromosomes in the fetal cells or determining a CNV profile associated with a genetic disorder or a predisposition of developing same.

Another embodiment contemplates an assay to detect a potential genetic abnormality in fetal cells, the method comprising:

(i) obtaining a sample from a pregnant female comprising both maternal and fetal cells;
(ii) optionally distributing the cells on a solid support if there is a microscopic component in the assay;
(iii) contacting the cells with two nucleic acid probes each labeled with distinguishable reporter molecules each capable of giving an identifiable signal wherein one probe is capable of hybridizing to both paternal and maternal chromosomal DNA and the other probe is capable of hybridizing to a portion of chromosomal DNA present in the paternal chromosome but deleted in the maternal chromosome;
(iv) determining whether a cell is a fetal or maternal cell based on the presence of a cell exhibiting a single signal (maternal cells) together with a combination of signals (fetal cells); and
(v) conducting a genetic test on the chromosomes in the fetal cells or enumerating the chromosomes in the fetal cells or determining a CNV profile associated with a genetic disorder or a predisposition of developing same.

In relation to aneuploidy or polyploidy, the present invention further contemplates an assay for detecting a chromosomal aneuploidy or polyploidy in a fetal cell, the method comprising:

(i) obtaining a sample from a pregnant female comprising both maternal and fetal cells;
(ii) optionally distributing the sample on a solid support if there is a microscopic component in the assay;
(iii) contacting the cells with a nucleic acid probe labeled with a reporter molecule capable of giving an identifiable signal which probe is capable of hybridizing to paternally-derived chromosomal DNA within a sequence which is absent in maternal chromosomal DNA;
(iv) determining which cells are maternal or fetal based on the presence of a signal wherein the presence of a signal wherein the presence of two signals is indicative of a fetal cell; and
(v) screening for chromosomal number based on the number of signals in fetal cells.

Another aspect of the present invention provides an assay for detecting a chromosomal aneuploidy or polyploidy in a fetal cell, the method comprising:

(i) obtaining a sample from a pregnant female comprising both maternal and fetal cells;
(ii) optionally distributing the sample on a solid support if there is a microscopic component in the assay;
(iii) contacting the cells with a nucleic acid probe labeled with a reporter molecule capable of giving an identifiable signal which probe is capable of hybridizing to paternally-derived chromosomal DNA at the flanking sequences of a deletion, which deletion is absent in paternal chromosomal DNA;
(iv) determining which cells are maternal or fetal based on the presence of two signals is indicative of a fetal cell; and
(v) screening for chromosomal number based on the number of signals in fetal cells.

Still another aspect of the present invention provides an assay for detecting a chromosomal aneuploidy or polyploidy in a fetal cell, the method comprising:

(i) obtaining a sample from a pregnant female comprising both maternal and fetal cells;
(ii) optionally distributing the sample on a solid support if there is a microscopic component in the assay;
(iii) contacting the cells with two nucleic acid probes each labeled with different reporter molecules each capable of giving an identifiable signal wherein one probe is capable of hybridizing to both maternal and paternal chromosomal DNA and the other probe is capable of hybridizing to a portion of chromosomal DNA present in the maternal chromosome but absent in the paternal chromosome;
(iv) determining which cells are maternal or fetal based on the presence of a signal wherein the presence of a single signal (maternal cells) together with a combination of signals (fetal cells).

Another aspect of the prevent invention provides an assay for detecting a chromosomal aneuploidy or polyploidy in a fetal cell, the method comprising:

(i) obtaining a sample from a pregnant female comprising both maternal and fetal cells;
(ii) optionally distributing the sample on a solid support if there is a microscopic component in the assay;
(iii) contacting the cells with two nucleic acid probes each labeled with different reporter molecules each capable of giving an identifiable signal wherein one probe is capable of hybridizing to both paternal and maternal chromosomal DNA and the other probe is capable of hybridizing to a portion of chromosomal DNA present in the paternal chromosome but absent in the maternal chromosome;
(iv) determining which cells are maternal or fetal based on the presence of a signal wherein the presence of a single signal (maternal cells) together with a combination of signals (fetal cells).

The “microscopic component” is the use of microscopy or other visual detection means to screen for cells or probe or signals or distribution on a solid support is less appropriate if the cells undergo FACS. In that case, this step is not used.

The term “nucleic acid” as used herein designates single- or double-stranded mRNA, RNA, cRNA, RNAi and DNA inclusive of cDNA, genomic DNA and DNA-RNA hybrids. Generally, the nucleic acid tested in the cells is chromosomal DNA.

A “probe” is usually a single-stranded or double stranded oligonucleotide, preferably having 100-1000 contiguous nucleotides which, for example, is capable of annealing to a complementary nucleic acid. Conveniently, a 5 to 100 kb DNA fragment is subjected to labeling which produces 100-1000 nucleotide labeled fragments. Examples of different sized fragments include 100, 200, 300, 400, 500, 600, 700, 800, 900 and 1000 bp fragments. A 400 bp fragment (+/−100 bp) is particularly useful in the practice of the present invention. Generally, the probe is suitably labeled with a reporter molecule capable of giving an identifiable signal. “Signals” include light waves, fluorescence, radio signals or other emissions. The probes hybridize to complementary regions of the chromosome (or mRNA) under particular stringency conditions. A “signal” includes a combination of signals such as a color generated by two other colors.

Reference herein to low stringency includes and encompasses from at least about 0 to at least about 15% v/v formamide (including 1%, 2%, 3%, 4%, 5%, 6%, 7%, 8%, 9%, 10% 11%, 12%, 13% and 14% v/v formamide) and from at least about 1 M to at least about 2 M salt for hybridization, and at least about 1 M to at least about 2 M salt for washing conditions. Generally, low stringency is at from about 25-30° C. to about 52° C., such as 25, 26, 27, 28, 29, 30, 31, 32, 33, 34, 35, 36, 37, 38, 39, 40, 41, 42, 43, 44, 45, 46, 47, 48, 49, 50, 51 and 52° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide, including 16%, 17%, 18%, 19%, 20%, 21%, 22%, 23%, 24%, 24%, 26%, 27%, 28%, 29% and 30% v/v formamide, and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C) % (Marmur and Doty, J. Mol. Biol. 5:109, 1962). However, the Tₘ of a duplex DNA decreases by 1° C. with every increase of 1% in the number of mismatch base pairs (Bonner and Laskey, Eur. J. Biochem. 46:83, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The target of the probe may be referred to as a “genetic marker” or “marker” or “deletion CNV” or “repeat CNV” which includes any locus or region of a genome. The genetic marker may be a coding or non-coding region of a genome. For example, genetic markers may be coding regions of genes, non-coding regions of genes such as introns or promoters, or intervening sequences between genes such as those that include polymorphisms, such as single nucleotide polymorphisms (SNPs), tandem repeat sequences, for example, satellites, microsatellites, short tandem repeats (STRs) and minisatellites, although without limitation thereto. Deletion CNV's are particularly useful, especially those associated with a phenotype or disease condition or which are useful for distinguishing between cell types. Deletion CNV's include deletions of from 1 kb to 100 Mb including 10, 20, 30, 40, 50, 60, 70, 80, 90 or 100 kb, 100, 200, 300, 400, 500, 600, 700, 800, 900 or 1,000 kb and 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 Mb. A “CNV” includes a copy number deletion and insertion.

Genetic analysis may be performed by any method including, but not limited to, fluorescence in situ hybridization (FISH), microscopy including scanning microscopy, primed in situ synthesis (PRINS) and nucleic acid sequence amplification, preferably in the form of multiplex fluorescent PCR amplification (MFPCR) or methods that employ nucleic acid arrays such as a microarray format.

It will be appreciated that genetic analysis may be performed using microarrays which are particularly useful when analyzing expression or non-expression of multiple genetic markers, mutation detection or polymorphisms in multiple genes or locations.

Reporter molecules providing a colored signal are particularly preferred. Colored signals include fluorescent signals.

Examples of fluorescent in situ hybridization (FISH) and Primed In Situ Synthesis (PRINS) may be found in Findlay et al, J. Assisted Reproduction & Genetics 15:257, 1998. In addition, labels listed in Table 1 are contemplated for use in the present invention.

TABLE 1

| | | |
|---|---|---|
| Probe | Ex¹ (nm) | Em² (nm) |

Reactive and conjugated probes

| | | |
|---|---|---|
| Hydroxycoumarin | 325 | 386 |
| Aminocoumarin | 350 | 455 |
| Methoxycoumarin | 360 | 410 |
| Cascade Blue | 375; 400 | 423 |
| Lucifer Yellow | 425 | 528 |
| NBD | 466 | 539 |
| R-Phycoerythrin (PE) | 480; 565 | 578 |
| PE-Cy5 conjugates | 480; 565; 650 | 670 |
| PE-Cy7 conjugates | 480; 565; 743 | 767 |
| APC-Cy7 conjugates | 650; 755 | 767 |
| Red 613 | 480; 565 | 613 |
| Fluorescein | 495 | 519 |
| FluorX | 494 | 520 |
| BODIPY-FL | 503 | 512 |
| TRITC | 547 | 574 |
| X-Rhodamine | 570 | 576 |
| Lissamine Rhodamine B | 570 | 590 |
| PerCP | 490 | 675 |
| Texas Red | 589 | 615 |
| Allophycocyanin (APC) | 650 | 660 |
| TruRed | 490, 675 | 695 |
| Alexa Fluor 350 | 346 | 445 |
| Alexa Fluor 430 | 430 | 545 |
| Alexa Fluor 488 | 494 | 517 |
| Alexa Fluor 532 | 530 | 555 |
| Alexa Fluor 546 | 556 | 573 |
| Alexa Fluor 555 | 556 | 573 |
| Alexa Fluor 568 | 578 | 603 |
| Alexa Fluor 594 | 590 | 617 |
| Alexa Fluor 633 | 621 | 639 |
| Alexa Fluor 647 | 650 | 688 |
| Alexa Fluor 660 | 663 | 690 |
| Alexa Fluor 680 | 679 | 702 |
| Alexa Fluor 700 | 696 | 719 |
| Alexa Fluor 750 | 752 | 779 |
| Cy2 | 489 | 506 |
| Cy3 | (512); 550 | 570; (615) |
| Cy3, 5 | 581 | 596; (640) |
| Cy5 | (625); 650 | 670 |
| Cy5, 5 | 675 | 694 |
| Cy7 | 743 | 767 |

Nucleic acid probes

| | | |
|---|---|---|
| Hoeschst 33342 | 343 | 483 |
| DAPI | 345 | 455 |
| Hoechst 33258 | 345 | 478 |
| SYTOX Blue | 431 | 480 |
| Chromomycin A3 | 445 | 575 |
| Mithramycin | 445 | 575 |
| YOYO-1 | 491 | 509 |
| SYTOX Green | 504 | 523 |
| SYTOX Orange | 547 | 570 |
| Ethidium Bormide | 493 | 620 |
| 7-AAD | 546 | 647 |
| Acridine Orange | 503 | 530/640 |
| TOTO-1, TO-PRO-1 | 509 | 533 |
| Thiazole Orange | 510 | 530 |
| Propidium Iodide (PI) | 536 | 617 |
| TOTO-3, TO-PRO-3 | 642 | 661 |
| LDS 751 | 543; 590 | 712; 607 |

Cell function probes

| | | |
|---|---|---|
| Indo-1 | 361/330 | 490/405 |
| Fluo-3 | 506 | 526 |
| DCFH | 505 | 535 |
| DHR | 505 | 534 |
| SNARF | 548/579 | 587/635 |

Fluorescent Proteins

| | | |
|---|---|---|
| Y66F | 360 | 508 |
| Y66H | 360 | 442 |
| EBFP | 380 | 440 |
| Wild-type | 396, 475 | 50, 503 |
| GFPuv | 385 | 508 |
| ECFP | 434 | 477 |
| Y66W | 436 | 485 |
| S65A | 471 | 504 |
| S65C | 479 | 507 |
| S65L | 484 | 510 |
| S65T | 488 | 511 |
| EGFP | 489 | 508 |
| EYFP | 514 | 527 |
| DsRed | 558 | 583 |

Other probes

| | | |
|---|---|---|
| Monochlorobimane | 380 | 461 |
| Calcein | 496 | 517 |

¹Ex: Peak excitation wavelength (nm)
²Em: Peak emission wavelength (nm)

These reporter molecules are generally attached to the nucleic acid probe. Standard chemistry is used to attach the reporter molecules. In this regard, terms such as “label”, “reporter molecule”, “signaling molecule” and the like are used interchangeably throughout the subject specification. The reporter molecule genes are a signal. Reference to the “signal” includes a combination of signals.

Rhodamine (red) and fluorescein (green) are particularly useful labels.

In accordance with the present invention, a combination of signals is detectable over the presence of a single signal. For example, a label emitting a red color and a label emitting a green color leads to a signal which may be yellow or a “blurring” of red and green. This is a combination of signals encompassed by the term “signal”.

The genetic testing may also include multiplexing such as multiplex amplification or multiplex PCR which refers to amplification of a plurality of genetic markers in a single amplification reaction. Alternatively, multiple hybridization reactions all with different reporter molecules may be used to identify or test for a range of genetic abnormalities.

Nucleic acid amplification techniques are well known to the skilled addressee, and also include ligase chain reaction (LCR) as for example described in Ausubel et al, Current protocols in molecular biology 15, John Wiley & Sons NY, 1995-1999, strand displacement amplification (SDA) as for example described in U.S. Pat. No. 5,422,252; rolling circle replication (RCR) as for example described in Liu et al, J. Am. Chem. Soc. 118:1587, 1996 and International Application WO 92/01813 and International Application WO 97/19193; nucleic acid sequence-based amplification (NASBA) as for example described by Sooknanan et al, Biotechniques 17:1077, 1994; and Q-(3 replicase amplification as for example described by Tyagi et al, Proc. Natl. Acad. Sci. USA 93:5395, 1996.

The above-mentioned are examples of nucleic acid sequences amplification techniques but are not presented as an exhaustive list of techniques. Persons skilled in the art will be well aware of a variety of other applicable techniques as well as variations and modifications to the techniques described herein.

Although the present invention also contemplates use of nucleic acid other than DNA, the particular nucleic acid is DNA.

More particularly, the nucleic acid is genomic DNA.

Isolation of cellular nucleic acids is well known in the art, although the skilled person is referred to Ausubel et al, Current protocols in molecular biology 2, 3, 4, John Wiley & Sons NY, 1995-1999, for examples of nucleic acid isolation.

The present invention further enables the detection of chromosome number in an organism. The present invention also has application for the detection of non-disjunction events in reproductive and non-reproductive cells and identification of trisomy. In this aspect of the present invention, cells of the subject, such as a human, may be tested for missing and/or duplicated chromosomes. The method of this aspect of the present invention would be largely similar to the methods hereinbefore described.

The present invention is further directed to kits, such as test kits, for isolating particular cell types and/or detecting genetic alterations, disorders or abnormalities in cells. The kit comprises compartments adapted to contain a solid support to receive a sample comprising maternal and fetal cells, nucleic acid probes as herein described, and reagents for recording or detecting reporter molecule signals such as fluorescence from the labels. The kits may also be interfaced with FACS machines, microscopic devices and/or cell collection devices.

The present invention is further directed to a panel of nucleic acid probes such as listed in a database of genomic variants. For example, http://projects.tccg.ca/variation or any updated version thereof lists a range of genomic variants which can be used to generate a microarray or panel of probes. Alternatively, the panel of probes may be used to identify probes that can then be used in a particular assay. For example, a cancer cell type can be identified by a CNV and then the appropriate probe used for subsequent analysis. Similarly, the panel of probes can be used to identify a suitable probe to monitor the fate of donor transplant cells, such as donor bone marrow transplant cells.

The Database of Genomic Variants (DGV) lists a total of 5672 copy number variable (CNV) regions. These data have been collected from a large number of independent studies on healthy individuals that have employed genome scans (i.e. array-CGH, SNP micro-array) for CNV detection.

In addition to the data in the DGV there are a number of large, co-operative, studies aimed at cataloguing CNV in large populations (2,000-20,000) of healthy individuals (Ionita-Laza et al, Bioinformatics, 2008; McCarroll et al, Nat Genet, 2008; Mefford et al, N Engl J Med, 2008).

It is now evident that there are a number of CNVs that are very common in the human genome. Some examples of these are given in Table 2. This is not by any means an exhaustive list as would be known to the skilled artisan.

TABLE 2
Examples of Common CNVs Ideal for Rare Cell Detection

| | | | | | Population |
|---|---|---|---|---|---|
| | | LocusStart | LocusEnd | FISH | Frequency |
| CND | Chr | (bp) | (bp) | Marker | (%) |
| 1 | chr14 | 104579618 | 106318151 | rp11-112h5 | 40 |
| 2 | chr9 | 38399006 | 46588540 | rp11-286g23 | 29 |
| 3 | chr6 | 78852599 | 79250187 | rp11-314b9 | 26 |
| 4 | chr1 | 16612540 | 17415709 | rp11-91d22 | 26 |
| 5 | chr16 | 31658070 | 34997000 | rp11-488i20 | 66 |
| 6 | chr11 | 54592199 | 55636002 | rp11-746c5 | 31 |
| 7 | chr19 | 59354929 | 60158136 | rp11-707o7 | 9 |
| 8 | chr9 | 65387631 | 67803880 | rp11-318k12 | 30 |
| 9 | chr19 | 183069 | 2051346 | rp11-75h6 | 19 |
| 10 | chr7 | 142857897 | 143999571 | rp11-157H3 | 68 |
| 11 | chr3 | 163666156 | 165466432 | rp11-33c10 | 55 |
| 12 | chr3 | 41866 | 734804 | rp11-838m18 | 35 |
| 13 | chr20 | 60216284 | 61895105 | rp11-93b14 | 14 |
| 14 | chr8 | 39293436 | 39716796 | rp11-97d17 | 62 |
| 15 | chr2 | 88846016 | 91638754 | rp11-1134e24 | 57 |
| 16 | chr5 | 96481913 | 97151719 | RP11-140E9 | 6 |
| 17 | chr10 | 46519875 | 47154881 | RP11-192A16 | 7 |
| 18 | chr2 | 242173260 | 242650580 | RP11-93H11 | 7 |
| 19 | chr8 | 137757137 | 137955330 | RP11-17M8 | 6 |
| 20 | chr10 | 27589967 | 27753531 | RP11-748L13 | 4 |
| 21 | chr1 | 194997796 | 195274102 | RP11-109P13 | 4 |
| 22 | chr20 | 14592859 | 14785965 | RP11-779J19 | 4 |
| 23 | chr21 | 30141891 | 30358836 | RP11-381F12 | 3 |

Chr—Chromosome
FISH—Fluorescence in situ Hybridization
CND—Copy Number deletion
CNDR—Copy Number Deletion Region. These regions are copy number variable in healthy individuals (general population). Any one individual can have between 0-3 copies of this segment of DNA in their cells.
CNDR 1-15: 15 of the most common copy number deletions reported in the DGV (http://projects.tcag.ca/variation/) as of the 20 Sep. 2008.
CNDR 16-23: 8 common copy number deletions identified in a local Australian population using Affymetrix (Trade Mark) 250K Nsp genome scans.
Population Frequency refers to the proportion of individuals in the studies population that carry 0 or 1 (i.e. deletion) copies of this region.

Fluorescently-labeled FISH probes have been prepared that target these CND regions. This strategy of using a panel of FISH probes targeting common CNV regions enables the identification of cells including rare cells in a mixture of cells that have been prepared on the surface of a microscope slide. In addition, it has the added benefit of avoiding the need to test parental samples as no prior knowledge of CNV status is required. In theory, cells are identifiable by the presence of an additional probe signal (copy). Essentially, cells are identified as harboring different signal patterns (be it number or color of signals, for example) compared to the majority of cells on the slide surface. By using a panel (n≧10; where the probes target regions with population frequencies >0.05) of common CNV probes the probability that at least one of these regions will be different in DNA copy number between the target cells and remaining cells is very high. Detection of the fluorescent probe signals is achieved by manual inspection of the slides using a fluorescent microscope. Manual detection of so many probe hybridizations/cells can be time-consuming and labor intensive. Alternatively, detection can be automated by use of robotic systems (Evans et al, Fetal Diagn Ther 21:523-7, 2006; Johnson et al, Microsc Res Tec 70:585-8, 2007; Ntouroupi et al, Br J Cancer 99:789-95, 2008; Seppo et, al, Prenat Diagn 28:815-21, 2008; Wauters et al, Prenat Diagn 27:951-5, 2007) that enable rapid scanning of the entire microscope slide (up to ˜100,000 cells), identification of cells harboring the signal pattern of interest (i.e. spot counting), and subsequent inspection by a human analyst.

Hence, the panel may be specific, for example, for CNVs associated with transplanted cells, organ or hemopoietic or neurologic cell types, or cancer cells.

Furthermore, the ability to detect rare cell types facilitates methods of treatment involving rare cells. For example, autologous or heterologous stem cells may be identified and the population expanded ex vivo before being re-introduced to the subject or another subject. In addition, using the CNV signature of the present invention, the fate and/or distribution of these stem cells can be followed and monitored.

Hence the present invention contemplates a method of treatment or monitoring a treatment, the method comprising collecting a sample comprising cells to be transplanted to a subject and contacting the cells with a nucleic acid probe labeled with a reporter molecule capable of giving an identifiable signal which probe is capable of hybridizing to chromosomal DNA at the flanking sequences of a deletion in one cell type, which deletion absent in chromosomal DNA in another cell type, wherein the presence or absence of a signal in a cell is indicative of a particular cell type expanding the desired cells ex vivo and then transplanting the cells into the same subject or a different subject and then using the same method to monitor distribution of said cells.

Another aspect of the present invention provides a method of treatment or monitoring a treatment, the method comprising collecting a sample comprising cells to be transplanted to a subject, contacting the cells with two nucleic acid probes each labeled with distinguishable reporter molecules each capable of giving an identifiable signal wherein one nucleic acid probe is capable of hybridizing to chromosomal DNA from one cell type whereas both nucleic acid probes are capable of hybridizing to a portion of chromosomal DNA present in another cell type, wherein the presence or absence of a single signal or a combination of signals in a cell is indicative of the cell types expanding the desired cells ex vivo and then transplanting the cells into the same subject or a different subject and then using the same method to monitor distribution of said cells.

Generally, the desired cells are stem cells. In a particular embodiment, the stem cells are autologous to the subject being treated. The subject may be a human or non-human animal.

The present invention is further illustrated by the following examples which should not be construed as further limiting. The contents of all figures are expressly incorporated herein by reference in relation to the Examples.

### Example 1

### Detection of Donor Cells in Bone Marrow Transplant Patients

A copy number deletion present in the transplant patient or recipient's DNA which is not present in the donor's DNA is detected to distinguish the donor cells in the recipient.

By using two color FISH test using a probe within and an adjacent probe outside the copy number deletion, the recipient non-deleted locus is one color such as yellow (red+green) and the copy number deletion is red. If the copy number deletion is not present, the donor cells show a signal which is two yellow signals (red+green).

The test is also done with only one color (e.g. red) using a probe within the recipient copy number deletion. Recipient cells show one red signal and the donor cells show two red signals. As false positive cells showing two signals are unlikely, i.e. Recipient cells masquerading as donor cells, this single color test is applicable here.

This test is not restricted to using host/recipient specific copy number deletions. It works equally well with donor specific copy number deletions.

250K SNP arrays with CNAG (Nannya et al, Cancer Res. 65(14):6071-6079, 2005) and AROMA (http://groups.google.com/group/aroma-affymetrix. Bengtsson H) analysis software is used to detect copy number deletions in the samples. The algorithms used by these two software programs are fundamentally different and provide a complementary analysis.

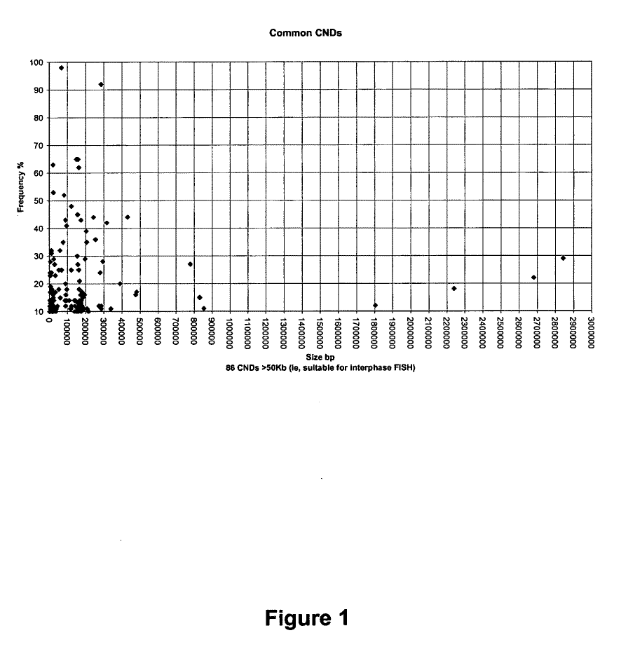

By analysing published data common CNV and using the panel of probes on donor material one determines probes which are potentially useful as they do not bind and therefore are useful to detect the donor cells. The probes for this experiment may include the use of a panel/set of common CNVs as per but not restricted to Table 2, selected from published data, to perform FISH on donor material. In this manner one can determine probes which will be potentially useful as they will not bind and therefore will be useful to detect the donor cells. There is a high degree of confidence that there will be no limitation on the number of copy number deletions available for testing. The current estimate using higher density arrays is 50 deletions of 10 Kb or larger per individual (see also FIG. 1).

### Example 2

### Detection of Down Syndrome

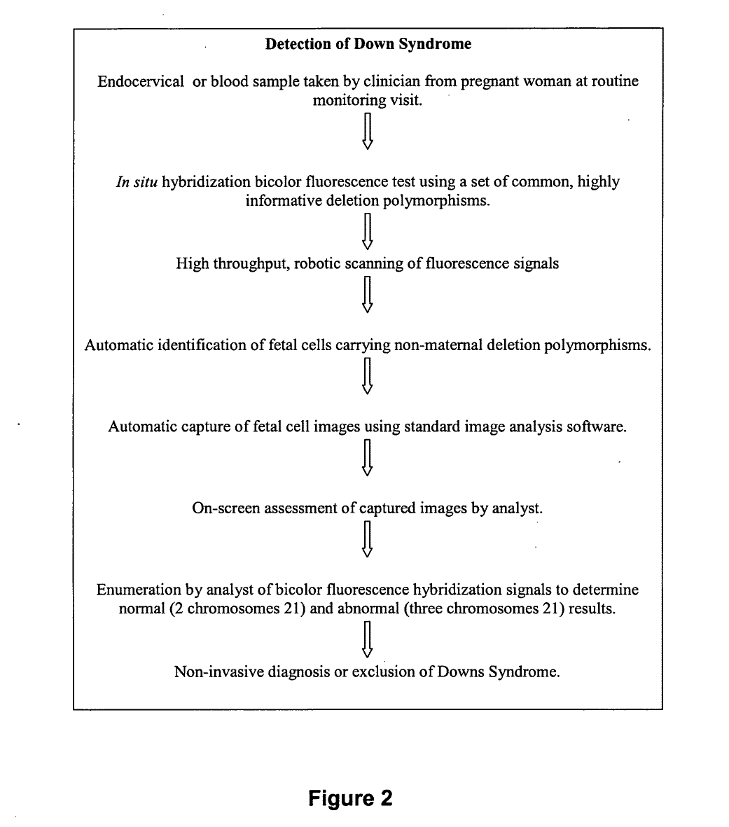

A schematic protocol for detecting Down Syndrome is shown in FIG. 2.

Individuals with Down Syndrome have trisomy 21, i.e. have three instead of two chromosomes 21. The assay is a non-invasive pre-natal test for trisomy 21, which is performed in situ on the microscope slide, thus avoiding the need to manually isolate fetal cells. The test is based on the analysis of a class of DNA polymorphism called Copy Number Variation (CNV). Fetal cells are distinguished from maternal cells through detection of non-maternal copy number variations. Specifically, deletion polymorphisms are used that have been inherited by the fetus from the father and are not carried by the mother. As only fetal cells carry these non-maternal polymorphisms, they can be distinguished from maternal cells. Deletion polymorphisms are located throughout the human genome. By using deletion polymorphisms located on chromosome 21 with the in situ assay, not only are fetal cells identified, but the number of chromosomes 21 present in these fetal cells can be enumerated to diagnose trisomy 21.

Cataloging efforts show that there are more than adequate numbers of suitable deletion polymorphisms in the population which can be used for this test.

As a model system, a locus is used, which has been accurately mapped in a patient with a deletion on chromosome 5. In situ hybridization probes have been made matching the sections of DNA sequence within and flanking the deletion. By labeling the “within” and “flanking” sequences with two different fluorescence colors and hybridizing the probes to the patient's cells, the intact locus on the normal chromosome and the deleted locus on the abnormal chromosome can be readily identified. This model is directly applicable to detection of deletion polymorphisms and enumeration of copy number.

The non-invasive Down Syndrome test using the in situ deletion polymorphism assay is available to all pregnant women. It is highly automatable through use of commercially available robotic fluorescence slide scanners.

### Example 3

### Detection of Cells from a Fetus with Down Syndrome Method 1

An endocervical sample is taken by a clinician from a pregnant woman at routine monitoring visit at approximately seven weeks gestation. Such a sample contains mainly maternal endothelial cells with small numbers of cells of fetal origin. An aliquot of the cell preparation is pipetted onto a standard glass microscopy slide and air dried at ambient temperature.

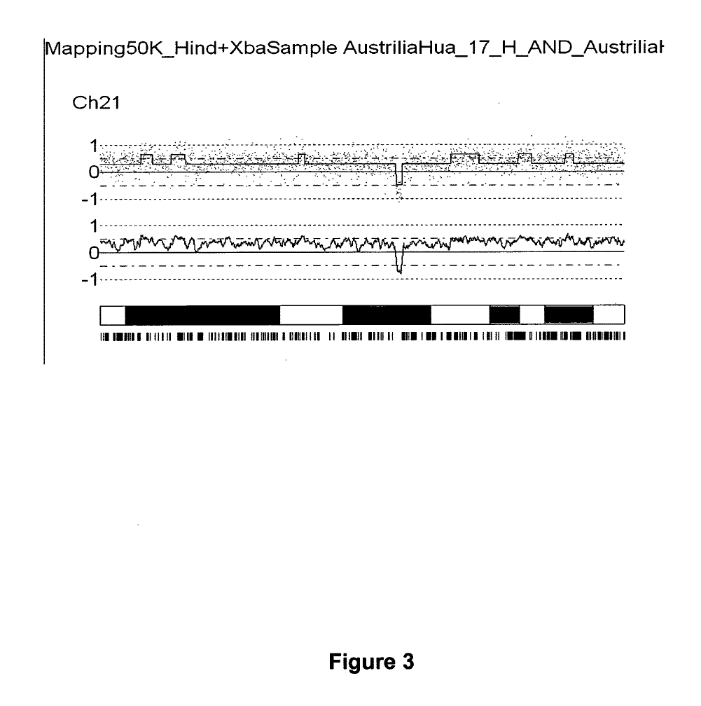

The Fluorescence in situ hybridization (FISH) test discriminates maternally and paternally derived DNA loci within the cells. Only fetal cells contain paternally derived loci and this is the basis on which fetal cells are distinguished from maternal cells. Any paternally derived locus is suitable for identification of fetal cells but those on chromosome 21 are most suitable for simultaneous assay of the number of chromosome 21s present for diagnosis of Down syndrome (see FIG. 3).

A locus is selected either by:

(1) prior knowledge of its presence through testing of the father's DNA; or
(2) use of a panel of approximately six to twelve loci as per but not restricted to Table 2 which are known to be common in the general population (taking into account any known racial differences) for which there is a high probability that at least one will be present in the paternally derived fetal DNA).

In relation to (1) the father's DNA is screened for known common deletions, which are normal Copy Number Variations (CNV) within his DNA. This is done using a multiplex, dosage-sensitive PCR assay of 20 known common loci on chromosome 21, selected from the Database of Common Variants: http://projects.tcag.ca/variation/. When a copy number deletion is identified in the father's DNA, a FISH test is designed using two probes, one of which is complimentary to the DNA sequence on one flank of the deletion and the other to the adjacent deleted sequence. The copy number deletion is approximately 100 kb to 200 kb in length and the probes are identified from a Bacterial Artificial Chromosome (BAC) clone library (http://genome.ucsc.edu), one clone specific for the human flanking sequence and one for the adjacent deletion sequence.

The flanking sequence BAC clone is labeled with directly-labeled Rhodamine (red fluorescence) and the deletion clone is labeled with directly-labeled Fluorescein (green fluorescence) by nick translation using a commercially available kit (Roche Inc).

The labeled probes are combined in approximately equal concentrations (50 ng/μl) in hybridization buffer (Vysis Inc) containing Cot1 DNA (1 μg/μl) for suppression of repetitive DNA sequences within the probes. The probes (2 μl) are pipetted onto the glass slide, covered with a round 12 mm coverslip, the edges of which are sealed with rubber sealant. The probe and cell DNA are co-denatured by heating the sealed slide for 3 minutes at 75° C. on a thermostated hotplate. The slide is then place in a sealed plastic box containing a damp towel to provide humidity. The box is placed in a 37° C. incubator overnight.

After incubation, the rubber sealant is removed with forceps and the slide placed coplin jar containing 2×SSC solution for 5 minutes at 73+/−2° C. to provide a stringency wash for removal of non-specifically bound probe. The slide is then stored in 4×SSC+0.1% v/v Tween 20 until ready for fluorescence detection.

Vectashield/DAPI mountant (8 μl, Sigma Inc) is pipetted onto the glass slide and a 24×60 mm coverslip placed on top. The slide is analysed using a fluorescence microscope (Zeiss Ltd) under 60-100× magnification with filters to identify DAPI, Rhodamine and Fluorescein simultaneously.

Cells containing two yellow or two overlapping clusters of red/green/yellow signals are maternal cells and are ignored.

Fetal cells are identified as those with at least one isolated red signal. These cells are further analysed to enumerate the number of signals observed, indicative of the number of chromosomes 21 present as follows:

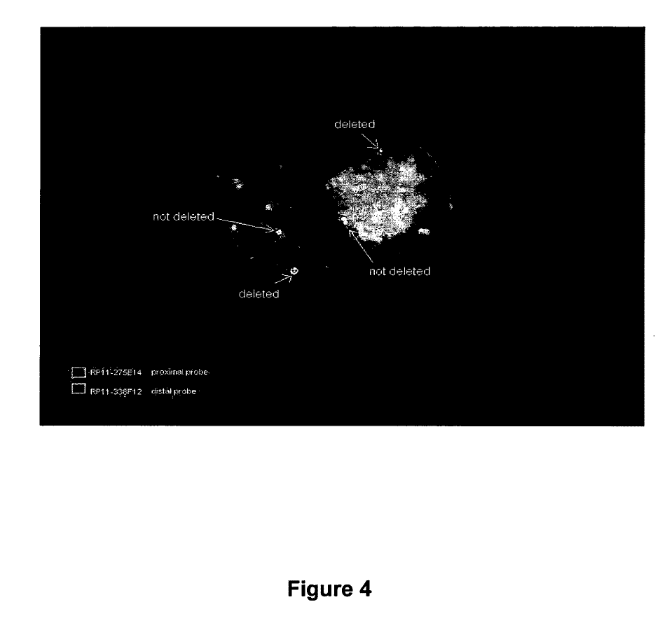

A cell with one isolated red signal and one yellow or one overlapping cluster of red/green/yellow signals is a fetal cell containing one paternally-derived targeted locus and one maternally-derived targeted locus, from which the presence of two copies of chromosome 21 is inferred (see FIG. 4).

A cell with one isolated red signal and two yellow or two overlapping clusters of red/green/yellow signals is a fetal cell containing one paternally-derived targeted locus and two maternally-derived targeted loci, from which the presence of three copies of chromosome 21 and Down syndrome is inferred.

A cell with two isolated red signals and one yellow or one overlapping cluster of red/green/yellow signals is a fetal cell containing two paternally-derived targeted loci and one maternally-derived targeted locus, from which the presence of three copies of chromosome 21 and Down syndrome is inferred.

### Example 4

### Detection of Cells from a Fetus with Down Syndrome Method 2

A copy number deletion present in the mother's DNA which is not present in the father's DNA is detected. As there is a 50% chance that the mother will not pass on such a copy number deletion to her offspring, fetal cells can be distinguished from maternal cells by the absence of the copy number deletion in the fetal cells. This has the advantage of not requiring analysis of paternal DNA and avoiding the possibility of non-paternity.

By using two color FISH test using a probe within and an adjacent probe outside the copy number deletion, the mother's non-deleted locus is yellow (red+green) and the copy number deletion will be red. If the copy number deletion has not been inherited by the fetus, fetal cells show two yellow signals (red+green).

The test can also be done with only one color (e.g. red) using a probe within the mother's copy number deletion. Maternal cells show one red signal and the fetal cells show two red signals. As false positive cells showing two signals are unlikely, i.e. maternal cells masquerading as fetal cells, this single color test is applicable here.

The assay is described in more detail below.

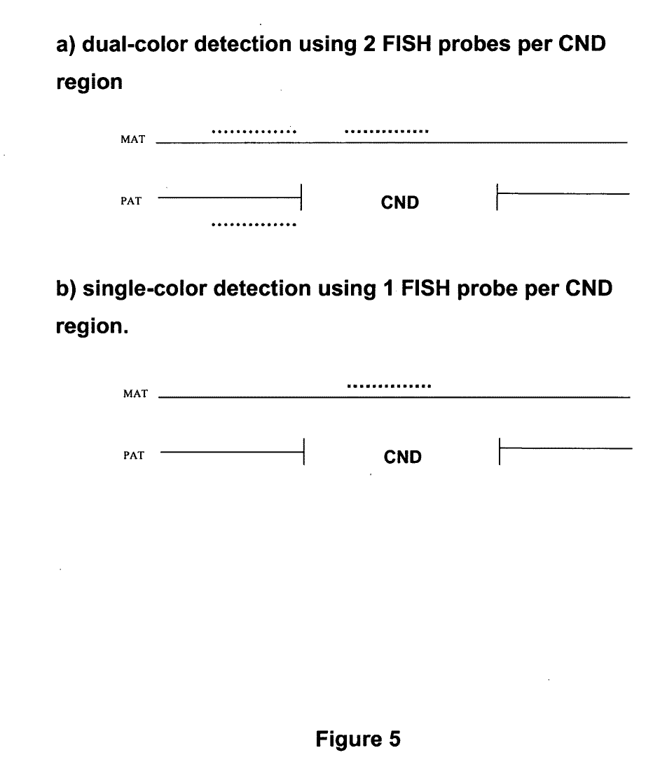

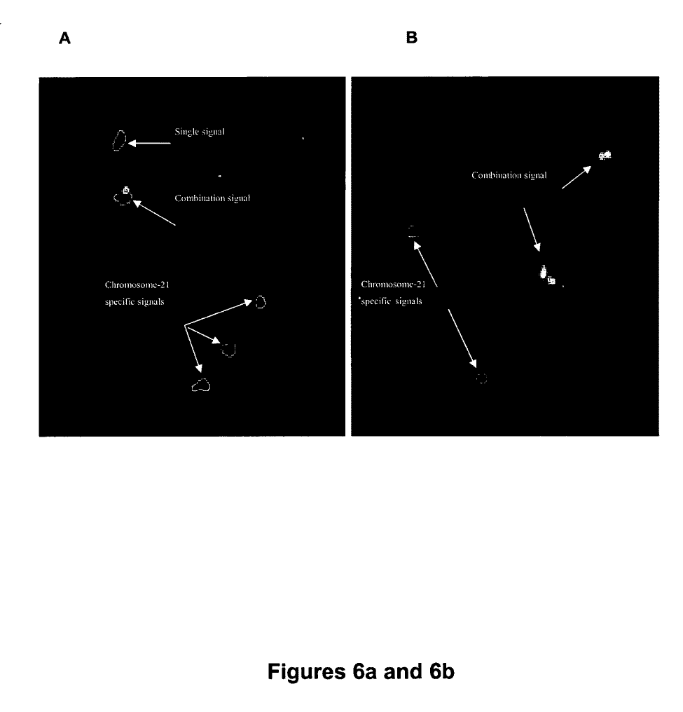

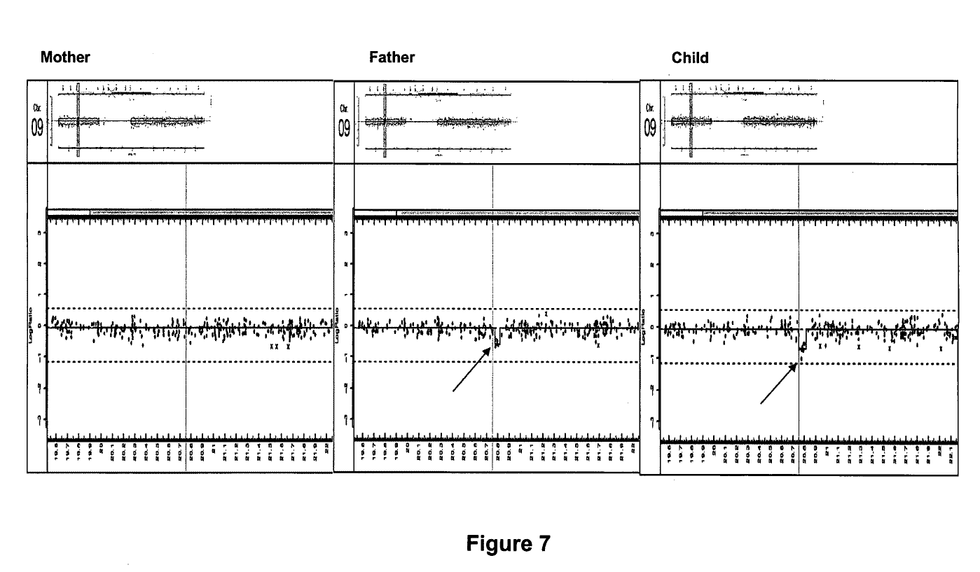

250K SNP arrays with CNAG (Nannya et al, Cancer Res. 65(14):6071-6079, 2005) and AROMA (http://groups.google.com/group/aroma-affymetrix. Bengtsson H) analysis software are used to detect copy number deletions in the samples (FIG. 7). The algorithms used by these two software programs are fundamentally different and provide a complementary analysis. The FISH probes are shown in FIG. 5. Spiking experiments are shown in FIGS. 6a and 6b.

By analysing published data of 30 CEPH family trios (McCarroll et al, Nat Genet. 2008 Sep. 7) using 6.0 SNP arrays indicate a median of 15 inherited paternal deletions per child. The median number of maternal copy number deletions not transmitted was 13. After restricting for CNV size (ie CNVs greater than 50 Kb) approximately 3-6 deletion polymorphisms would have been informative by CND-FISH. There is a high degree of confidence that there will be no limitation on the number of copy number deletions available for testing. The current estimate using higher density arrays is 50 deletions of 10 Kb or larger per individual (FIG. 7).

These data were gathered using 250K Nsp arrays and is likely to be an under-estimate of the ‘true’ level of copy number variations in these individuals. The latest release of SNP arrays (Affymetrix 6.0 chip) includes 900,000 SNPs that span the human genome and also include 946,000 (non-polymorphic) probes for the detection of copy number variation.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Ausubel et al, Current protocols in molecular biology 2, 3, 4, John Wiley & Sons NY, 1995-1999
Ausubel et al, Current protocols in molecular biology 15, John Wiley & Sons NY, 1995-1999
Bonner and Laskey, Eur. J. Biochem. 46:83, 1974
Evans et al, Fetal Diagn Ther 21:523-7, 2006
Findlay et al, J. Assisted Reproduction & Genetics 15:257, 1998
Ionita-Laza et al, Bioinformatics, 2008
International Application WO 92/01813
International Application WO 97/19193
Johnson et al, Microsc Res Tec 70:585-8, 2007
Liu et al, J. Am. Chem. Soc. 118:1587, 1996
McCarroll et al, Nat Genet, 2008
Marmur and Doty, J. Mol. Biol. 5:109, 1962
Mefford et al, N Engl J Med, 2008
Nannya et al, Cancer Res. 65(14):6071-6079, 2005
Ntouroupi et al, Br J Cancer 99:789-95, 2008
Redon et al, Nature 444(7118):444-454, 2006
Seppo et al, Prenat Diagn 28:815-21, 2008
Sooknanan et al, Biotechniques 17:1077, 1994
Tyagi et al, Proc. Natl. Acad. Sci. USA 93:5395, 1996
U.S. Pat. No. 5,422,252
Wauters et al, Prenat Diagn 27:951-5, 2007

1. A method for identifying a cell type in a sample comprising a mixture of cell types from a subject, said method selected from the list consisting of:
(i) collecting the sample with the cells and contacting the cells with a nucleic acid probe labeled with a reporter molecule capable of giving an identifiable signal which probe is capable of hybridizing to chromosomal DNA at the flanking sequences of a deletion in one cell type, which deletion is absent in chromosomal DNA in the other cell type wherein the presence or absence of a signal in a cell is indicative of a particular cell type; and
(ii) collecting the sample with the cells and contacting the cells with two nucleic acid probes each labeled with distinguishable reporter molecules each capable of giving an identifiable signal wherein one nucleic acid probe is capable of hybridizing to chromosomal DNA in one cell type whereas both nucleic acid probes are capable of hybridizing to a portion of chromosomal DNA in another cell, wherein the presence or absence of a single signal or a combination of signals in a cell is indicative of a particular cell type.

2. (canceled)

3. The method of claim 1 wherein the nucleic acid probe or probes detects the presence or absence of a copy number variation (CNV) selected from a deletion and an insertion.

4. (canceled)

5. The method of claim 3 further comprising sorting one cell type from another cell type by FACS.

6. The method of claim 3 wherein the cell types are monitored by scanning microscopy or other visual detection means.

7. The method of claim 6 wherein the sample with the cell is distributed on a solid support prior to application of the probes.

8. The method of claim 1 wherein the cells are derived from a transplant patient.

9. The method of claim 8 wherein the transplant patient is a bone marrow transplant patient.

10. The method of claim 1 wherein the cells are derived from a cancer patient or a subject being screened for cancer.

11. The method of claim 1 wherein the cells are derived from a pregnant female subject.

12. The method of claim 11 wherein the subject is a human.

13. The method of claim 11 or 12 wherein the cells are contacted with a single nucleic acid probe labeled with a reporter molecule capable of giving an identifiable signal which probe is capable of hybridizing to paternally-derived or maternally-derived chromosomal DNA at the flanking sequences of a deletion, which deletion is absent in maternal or paternal chromosomal DNA.

14. The method of claim 11 or 12 wherein the cells are contacted with two nucleic acid probes each labeled with different reporter molecules each capable of giving an identifiable signal wherein one probe is capable of hybridizing to both maternal and paternal chromosomal DNA and the other probe is capable of hybridizing to a portion of chromosomal DNA present in the maternal chromosome but deleted in the paternal chromosome.

15. The method of claim 1 further comprising subjecting the cells to genetic testing.

16-38. (canceled)

39. A method of treatment or monitoring a treatment, the method comprising collecting a sample comprising cells to be transplanted to a subject and contacting the cells with a nucleic acid probe labeled with a reporter molecule capable of giving an identifiable signal which probe is capable of hybridizing to chromosomal DNA at the flanking sequences of a deletion in one cell type, which deletion absent in chromosomal DNA in another cell type, wherein the presence or absence of a signal in a cell is indicative of a particular cell type expanding the desired cells ex vivo and then transplanting the cells into the same subject or a different subject and then using the same method to monitor distribution of said cells.

40. A method of treatment or monitoring a treatment, the method comprising collecting a sample comprising cells to be transplanted to a subject, contacting the cells with two nucleic acid probes each labeled with distinguishable reporter molecules each capable of giving an identifiable signal wherein one nucleic acid probe is capable of hybridizing to chromosomal DNA from one cell type whereas both nucleic acid probes are capable of hybridizing to a portion of chromosomal DNA present in another cell type, wherein the presence or absence of a single signal or a combination of signals in a cell is indicative of the cell types expanding the desired cells ex vivo and then transplanting the cells into the same subject or a different subject and then using the same method to monitor distribution of said cells.

41. (canceled)

42. An assay to detect a potential genetic abnormality in fetal cells said method selected from the list consisting of:
(A)
(i) obtaining a sample from a pregnant female comprising both maternal and fetal cells;
(ii) contacting the cells with a nucleic acid probe labeled with a reporter molecule capable of giving an identifiable signal which probe is capable of hybridizing to paternally-derived chromosomal DNA at the flanking sequences of a deletion, which deletion is absent in maternal chromosomal DNA;
(iii) determining which cells are maternal or fetal based on the presence of a signal wherein the presence of a signal from the reporter molecule is indicative of a fetal cell; and
(iv) screening for chromosomal number based on the number of signals in fetal cells; and
(B)
(i) obtaining a sample from a pregnant female comprising both maternal and fetal cells;
(ii) contacting the cells with two nucleic acid probes each labeled with different reporter molecules each capable of giving an identifiable signal wherein one probe is capable of hybridizing to both maternal and paternal chromosomal DNA and the other probe is capable of hybridizing to a portion of chromosomal DNA present in the maternal chromosome but absent in the paternal chromosome;
(iii) determining which cells are maternal or fetal based on the presence of a signal wherein the presence of a single signal (fetal cells) from the reporter molecule together with a combination of signals (maternal cells); and
(iv) screening for chromosomal number based on the number of signals in fetal cells.

43. The method of claim 15 wherein the genetic test comprises determining a CNV profile associated with a genetic disorder or a predisposition of developing same.

44. The method of claim 15 or 43 wherein the cells are tested for aneuploidy or polyploidy or any syndrome with an established aetiology of segmented copy number abnormality of chromosome 21.

45. The method of claim 44 wherein the fetal cells are tested for trisomy of chromosome 21.
